(19) United States
(12) Patent Application Publication
(10) Pub. No.: US 2025/0251346 A1
(43) Pub. Date: Aug. 7, 2025

(54) RAMAN HYPERSPECTROSCOPY OF SALIVA AND MACHINE LEARNING FOR SJOGREN'S SYNDROME DIAGNOSTICS

(71) Applicant: The Research Foundation for the State University of New York, Albany, NY (US)

(72) Inventors: Igor Lednev, Albany, NY (US); Melinda Larsen, Albany, NY (US); Neil Gildener-Leapman, Albany, NY (US); Bhavikkumar Vyas, Albany, NY (US)

(21) Appl. No.: 19/045,822

(22) Filed: Feb. 5, 2025

Related U.S. Application Data
(60) Provisional application No. 63/550,239, filed on Feb. 6, 2024.

(51) Int. Cl. G01N 21/65; G01N 33/487

(52) U.S. Cl. CPC G01N 21/65; G01N 33/48792; G01N 2201/1293; G01N 2201/1296

(57) ABSTRACT

A system and method for detecting Sjogren's Syndrome disease (SjD) in humans are disclosed. A sample, such as saliva, is obtained from a human subject and subjected to at least a Raman hyperspectroscopic analysis to produce a sample spectroscopic signature. The produced sample spectroscopic signature is analyzed using a predetermined statistical model based on spectroscopic signatures for a plurality of modeling samples, with the spectroscopic signatures for each of the plurality of modeling samples associated with SjD.

### CROSS-REFERENCE TO RELATED APPLICATION

This invention claims the benefit of U.S. Provisional Patent Application No. 63/550,239, filed on Feb. 6, 2024, the entirety of which is hereby incorporated herein by this reference.

### BACKGROUND OF THE INVENTION

### 1. Field of the Invention

The present invention generally relates to systems and methods for detecting human diseases and impairments. More particularly, the present invention relates to a system and method for detecting Sjogren's Syndrome disease (SjD) through Raman spectroscopy.

### 2. Description of the Related Art

Sjogren's Syndrome disease (SjD) is a chronic autoimmune disorder characterized by salivary and lacrimal gland damage, mediated by the immune system, leading to mouth and eye dryness stemming from salivary gland and lacrimal gland hypofunction, respectively. SjD is a systemic disease that primarily affects the exocrine organs, can have pleomorphic clinical presentations, and as such, have a significant impact on a patient's quality of life. SjD can exist in a “primary” form if it is not associated with other diseases or “secondary” if it occurs concurrently with another autoimmune disorder such as Rheumatoid Arthritis.

SjD affects middle-aged women significantly more than men, with the average female-to-male ratio being 9:1, irrespective of race and geographic location. Although the diagnosis is often made later in life, with a mean age of 52 to 62 years, the first symptoms may arise much earlier. Like most autoimmune diseases, the exact etiology of SjD is unclear. Currently, the most widely accepted theory centers around exposure to environmental factors, especially viruses such as the Epstein-Barr virus, which can cause dysregulation of the immune system.

The most common symptoms in SjD patients are ocular and mouth dryness [2]. Decreased saliva production often presents as dysphagia and dysgeusia, with difficulty swallowing dry foods and speaking for a prolonged period. Physical examination of patients with SjD typically demonstrates dry, erythematous oral mucosa, often with dental caries or periodontal disease. Chronic enlargement of a major salivary gland is also frequent. In addition, low production of tears can lead to chronic ocular surface inflammation with signs such as photosensitivity, itching, and erythema. Symptoms related to other gland dysfunctions, such as respiratory tract and skin dryness, can also occur in some patients. These symptoms lead to a significant decline in quality of life for SjD patients.

Classification of SjD is complex and controversial. Although the American College of Rheumatology (ACR) and the European League Against Rheumatism (EULAR) have agreed on a set of criteria that were revised most recently in 2016, the criteria are complex and require a score of 4 from 5 tests. Some of the diagnostic tools currently employed include the presence of antinuclear antibodies, including Ro/SSA and La/SSB antibodies, but the presence of antibodies alone is insufficient to diagnose SjD, and not all patients have both antibodies. Other tests include an invasive salivary gland biopsy to identify focal lymphocytic sialadenitis and the presence of germinal centers and a measurement of salivary flow rate. In addition, patients are referred to an ophthalmologist to assess their lacrimal production via Schirmer's test and check the integrity of the epithelial layers of the cornea and conjunctiva via ocular staining. No single evidence-based standardized screening test can diagnose SjD patients who complain of dry mucous membranes. Because of the complexity of diagnosis and differing symptoms of patients, there is continued underdiagnosis of the disease, limiting the ability to provide therapy early in the disease or even appropriately recruit patients to clinical trials.

Raman Spectroscopy (RS) of saliva has shown promising results in diagnosing various cancers, viral infections as well as autoimmune diseases like Alzheimer's disease. Raman spectroscopy (RS) is a technique based on inelastic light scattering, which probes the total (bio) chemical composition of the sample. Recent scientific literature has demonstrated the potential of integrating Raman spectroscopy with machine learning techniques to distinguish individuals with SjD from healthy individuals, utilizing human blood samples.

Saliva is an “ultra-filtrate” of blood and can reflect many pathological states. Saliva collection is painless, non-invasive, and can be accomplished by the patient without a doctor's visit. The ease of collecting saliva makes possible continued monitoring or patients over time. Raman spectroscopy can probe the total biochemical composition of a saliva sample.

### BRIEF SUMMARY OF THE INVENTION

Briefly described, the present system and method utilize Machine learning utilizes a complex Raman hyperspectral dataset to generate a spectral “fingerprint” of the disease, potentially including contributions from several biomarkers. Raman hyperspectroscopy of saliva can be used with machine learning for differentiating SjD patients from healthy control (HC) individuals and other individuals with similar symptoms, such as those treated with radiation therapy for head and neck cancers (RD), as these patients also suffer from salivary hypofunction and xerostomia. Raman hyperspectroscopy can be used to differentiate between SjD, HC, and RD patients using a rapid, noninvasive saliva test.

In one embodiment, the invention provides a method for detecting SjD that includes providing a saliva sample from a human subject, subjecting at least a portion of the saliva sample to a Raman hyperspectroscopic analysis to produce a sample spectroscopic signature for the saliva sample, analyzing the produced sample spectroscopic signature using a predetermined statistical model, the predetermined statistical model based on spectroscopic signatures for a plurality modeling samples, wherein the spectroscopic signatures for each of the plurality of modeling samples are associated with SjD, and correlating the produced sample spectroscopic signature with one of the plurality of predetermined statistical models based on the spectroscopic signatures for each of the plurality of modeling samples of the predetermined statistical model.

In one embodiment, the invention provides a system for detecting SjD that includes a spectroscopy device subjecting at least a portion of a saliva sample from a human to a Raman hyerspectroscopic analysis to produce a sample spectroscopic signature for the saliva sample. There is at least one computing device in operable communication with the spectroscopy device, the at least one computing device configured to detect a SjD in the human subject by analyzing the produced sample spectroscopic signature using a predetermined statistical model, the predetermined statistical model based on spectroscopic signatures for a plurality modeling samples, wherein the spectroscopic signatures for each of the plurality of modeling samples are associated with SjD, and correlating the produced sample spectroscopic signature with SjD based on the spectroscopic signatures for each of the plurality of modeling samples of the predetermined statistical model.

The present invention therefore provides an advantage in more accurately and non-invasively diagnosing SjD. The present invention is also industrially applicable in the manufacture and use of medical diagnostic equipment. Other objects, features and advantages of the present invention will be apparent to one of skill in the art after review of the present application.

### BRIEF DESCRIPTION OF THE DRAWINGS

FIG. 1 is a schematic view of one embodiment of a system for detecting cognitive diseases and impairments in humans.

FIG. 2 is a schematic view of one embodiment of a computing system configured to detecting cognitive diseases and impairments in humans.

FIG. 3 is a table of Information about the donors' age and sex for Healthy control (HC), Radiation (RD), and Sjogren syndrome patients (SjD).

FIG. 4 is table of the assignments of the main Raman bands for human saliva samples.

FIG. 5A is a table of cross-validation predictions for individual spectra collected for samples in the calibration dataset.

FIG. 5B is a table of the performance matrix of SVM_DA cross validation at spectral level.

FIG. 6 is a graph 60 of the pre-processed mean Raman spectra of saliva acquired from Healthy controls (HC), Radiation therapy patients (RD) and Sjogren syndrome patients (SjD). Areas selected by Genetic Algorithm are highlighted (transparent grey).

FIG. 7A is a graph of difference spectrum between SjD and HC mean spectra, and one standard spectral deviation of SjD and HC spectra.

FIG. 7B is a graph of a difference spectrum between mean spectra of RD and HC, and standard spectral deviation of RD and HC.

FIG. 8 is a graphs of external validation of the SVM_DA model. The percent spectra assigned to HC (1), RD (2), and SjD (3) classes are reported for individual samples.

FIG. 9 is a graph 90 illustrating the outlier removal using Hoteling T2.

### DETAILED DESCRIPTION OF THE INVENTION

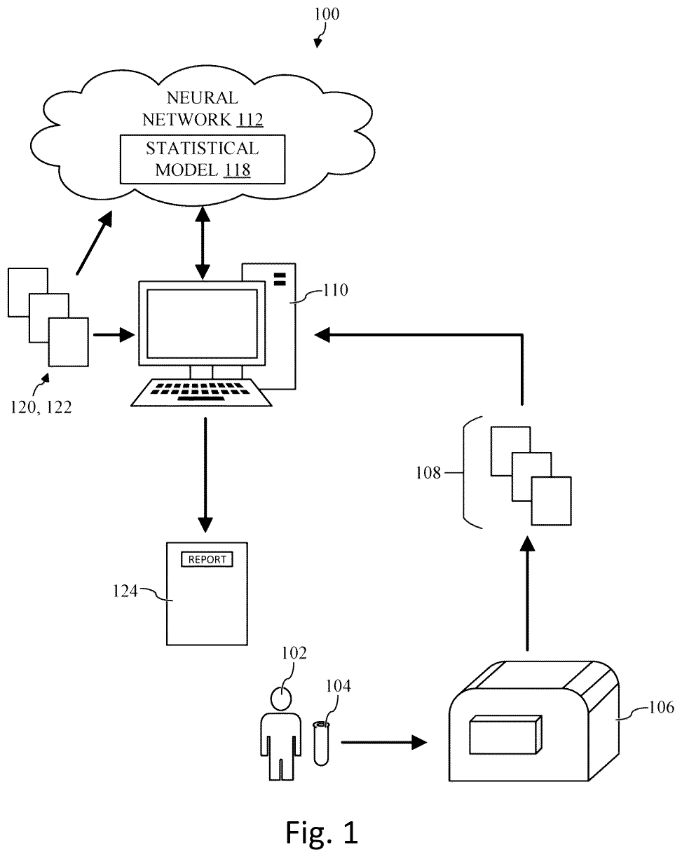

With reference to the figures in which like numerals represent like elements throughout the several views, FIG. 1 shows a non-limiting example of system 100 that may detect cognitive diseases and/or mental impairments in humans/human subjects 102 using saliva samples 104 as human biological samples. Almost any biolocial sample can be used here, such as blood, urine, sputum, etc., can be used, but saliva is preferred due to ease of collection. System 100 may include a spectroscopy device 106 configured to analyze saliva samples 104. That is, system 100 may include spectroscopy device 106 configured to subject at least a portion of a saliva sample 104 from a human 102 to a spectroscopic analysis to produce a sample spectroscopic signature 108 for the saliva sample 104.

In one example, only a single portion of saliva sample 104 may be analyzed, processed, and/or examined by spectroscopy device 106 to generate a single sample spectroscopic signature 108 for additional processing by system 100. In another non-limiting example, a plurality of portions or substantially the entirety of sample 104 from human 106 may be analyzed, processed, and/or examined by spectroscopy device 106 to generate a plurality of sample spectroscopic signatures 108 for further processing by system 100. That is, a plurality of portions of saliva sample 104 may be subject to spectroscopic analysis by spectroscopy device 106 to produce a plurality of distinct sample spectroscopic signatures 108 for saliva sample 104. Each of the plurality of portions of saliva sample 104 undergoing the spectroscopic analysis may be positionally distinct from the others in saliva sample 104.

In a non-limiting example, the spectroscopic analysis performed on saliva sample 104 using spectroscopic device 106 may include performing Raman spectroscopy. The Raman spectroscopy process may include, but may not be limited to: near-infrared (NIR) Raman spectroscopy, Raman microspectroscopy, Surface Enhanced Raman spectroscopy (SERS), surface enhanced resonance Raman spectroscopy (SERRS), Raman hyper spectroscopy, Fourier transform Raman spectroscopy, and coherent anti-Stokes Raman Spectroscopy (CARS).

Raman spectroscopy is a spectroscopic technique which relies on inelastic or Raman scattering of monochromatic light to study vibrational, rotational, and other low-frequency modes in a system (Gardiner, D. J., Practical Raman Spectroscopy, Berlin: Springer-Verlag, pp. 1-3 (1989), which is hereby incorporated by reference in its entirety). Vibrational modes are very important and very specific for certain types of chemical groups in molecules. They provide a “fingerprint” by which a molecule or biomolecule can be identified. The Raman effect is obtained when a photon interacts with the electron cloud of a molecule, exciting the electrons into a virtual state. The scattered photon is shifted to lower frequencies (Stokes process) or higher frequencies (anti-Stokes process) as it releases energy to or from the molecule, respectively. The polarizability change in the molecule will determine the Raman scattering efficiency, while the Raman shift will be equal to the energy (frequency) of the vibrational mode involved.

Fluorescence interference is the largest problem with Raman spectroscopy and is perhaps the reason why the latter technique has not been more popular in the past. If a sample contains molecules that fluoresce, the broad and much more intense fluorescence peak will mask the sharp Raman peaks of the sample. There are a few remedies to this problem. One solution is to use deep ultraviolet (DUV) light for exciting Raman scattering (Lednev I. K., “Vibrational Spectroscopy: Biological Applications of Ultraviolet Raman Spectroscopy,” in: V. N. Uversky, and E. A. Permyakov, Protein Structures, Methods in Protein Structures and Stability Analysis (2007), which is hereby incorporated by reference in its entirety). Practically no condensed face exhibits fluorescence below “250 nm. Possible photodegradation of biological samples is an expected disadvantage of DUV Raman spectroscopy. Another option to eliminate fluorescence interference is to use a near-IR (NIR) excitation for Raman spectroscopic measurement. Finally, surface enhanced Raman spectroscopy (SERS) which involves a rough metal surface can also alleviate the problem of fluorescence (Thomas et al., “Raman Spectroscopy and the Forensic Analysis of Black/Grey and Blue Cotton Fibers Part 1: Investigation of the Effects of Varying Laser Wavelength,” Forensic Sci. Int. 152:189-197 (2005), which is hereby incorporated by reference in its entirety). However, this method requires direct contact with the analyte and cannot be considered to be nondestructive.

Basic components of a Raman spectrometer are (i) an excitation source; (ii) optics for sample illumination; (iii) a single, double, or triple monochromator; and (iv) a signal processing system consisting of a detector, an amplifier, and an output device.

Typically, a sample is exposed to a monochromatic source usually a laser in the visible, near infrared, or near ultraviolet range. The scattered light is collected using a lens and is focused at the entrance slit of a monochromator. The monochromator, which is set for a desirable spectral resolution rejects the stray light in addition to dispersing incoming radiation. The light leaving the exit slit of the monochromator is collected and focused on a detector (such as a photodiode arrays (PDA), a photomultiplier (PMT), or charge-coupled device (CCD)). This optical signal is converted to an electrical signal within the detector. The incident signal is stored in computer memory for each predetermined frequency interval. A plot of the signal intensity as a function of its frequency difference (usually in units of wavenumbers, cm-1) will constitute the Raman spectroscopic signature (e.g., sample spectroscopic signature 108).

Raman signatures are sharp and narrow peaks observed on a Raman spectrum. These peaks are located on both sides of the excitation laser line (Stoke and anti-Stoke lines). Generally, only the Stokes region is used for comparison (the anti-Stoke region is identical in pattern, but much less intense) with a Raman spectrum of a known sample. A visual comparison of these set of peaks (spectroscopic signatures) between experimental and known samples is needed to verify the reproducibility of the data. Therefore, establishing correlations between experimental and known data is required to assign the peaks in the molecules and identify a specific component in the sample.

In another non-limiting example, vibrational spectroscopy may be used in system 100. In the non-limiting example, vibrational spectroscopy may include or involve Infrared (IR) absorption, Fourier Transform Infrared absorption (FTIR), Attenuated Total Reflection (ATR) FTIR or IR reflection spectroscopy. In this example where vibrational spectroscopy may be used, generated sample spectroscopic signature 108 may include a vibrational signature of saliva sample 104.

Fourier transform infrared (FTIR) spectroscopy is a versatile tool for the detection and structural determination of organic and inorganic compounds. In infrared spectroscopy (IR), IR radiation is passed through a sample. Some of the infrared radiation is absorbed by the sample and some is transmitted. The resulting spectrum represents a fingerprint of a sample with absorption peaks which correspond to the frequencies of vibrational modes of the material. Basic components of FTIR are (i) a light source; (ii) an interferometer; (iii) a sample; (iv) a detector; (v) computer.

Infrared radiation is emitted from a glowing black-body source. This beam passes through an aperture which controls the amount of energy presented to the sample (and, ultimately, to the detector). The beam enters the interferometer where “spectral encoding” takes place. The resulting interferogram signal then exits the interferometer. The beam enters the sample compartment where it is transmitted through or reflected off of the surface of the sample; depending on the type of analysis being accomplished, this is where specific frequencies, which are uniquely characteristic of the sample, are absorbed. The beam finally passes to the detector for final measurement. The measured signal is digitized and sent to the computer where the Fourier transformation takes place.

Microscopic-attenuated total reflectance (ATR) Fourier transform infrared (FT-IR) analysis is nondestructive, with analysis times competitive to current methodologies, and offers a molecular or biomolecule “fingerprint” of the analyzed sample. The vibrational signatures collected from the sample are easily discernible by the naked eye. Furthermore, this vibrational “fingerprint” targets a wider range of chemicals as compared to current methodologies, increasing the selectivity of the method. The optics of ATR-FT-IR imaging provides pseudo-immersion analysis. The high refractive index of the germanium ATR crystal increases the numerical aperture of the optics, enhancing spatial resolution by a factor of 4, without the use of a synchrotron light source (ATR accessories, An overview, PerkinElmer Life and Analytical Sciences, (2004), which is hereby incorporated by reference in its entirety). Exhaustive research applying micro-ATR-FT-IR chemical imaging (mapping) to the fields of bio-medical (Chan et al., Appl. Spectrosc. 59:149 (2005); Anastassopoulou et al., Vibrational Spectroscopy, 51:270 (2009); Kazarian et al., Biochimica et Biophysica Acta (BBA)-Biomembranes, 1758:858 (2006); Kazarian et al., Analyst, 138:1940 (2013), which are hereby incorporated by reference in their entirety) and forensic research (Dirwono et al., Forensic Science International, 199:6 (2010); Ng et al., Anal. and Bioanal. Chem. 394:2039 (2009); Spring et al., Anal. and Bioanal. Chem., 392:37 (2008), which are hereby incorporated by reference in their entirety) have been reported.

As discussed herein, subjecting saliva sample 104 to spectroscopic analysis via spectroscopic device 106 to generate sample spectroscopic signatures 108 may include for example, exposing biomolecules of saliva sample 104 to a spectroscopic analysis generated/performed by spectroscopy device 106. The exposure of biomolecules may, at least in part, contribute to the generation of sample spectroscopic signatures 108 and/or form the “fingerprinting” type of information included in produced signatures 108, as discussed herein. For example, at least one of the structural properties, conformational properties, or compositional variations of the exposed biomolecules may define the produced sample spectroscopic signature 108 for each saliva sample 104 analyzed by spectroscopy device 106. In non-limiting examples, the biomolecules that may aid in the generation and/or production of spectroscopic signature 108 may include, but are not limited to, proteins, lipids, peptides, amino acids, electrolytes, mucus, enzymes, and/or antibacterial species.

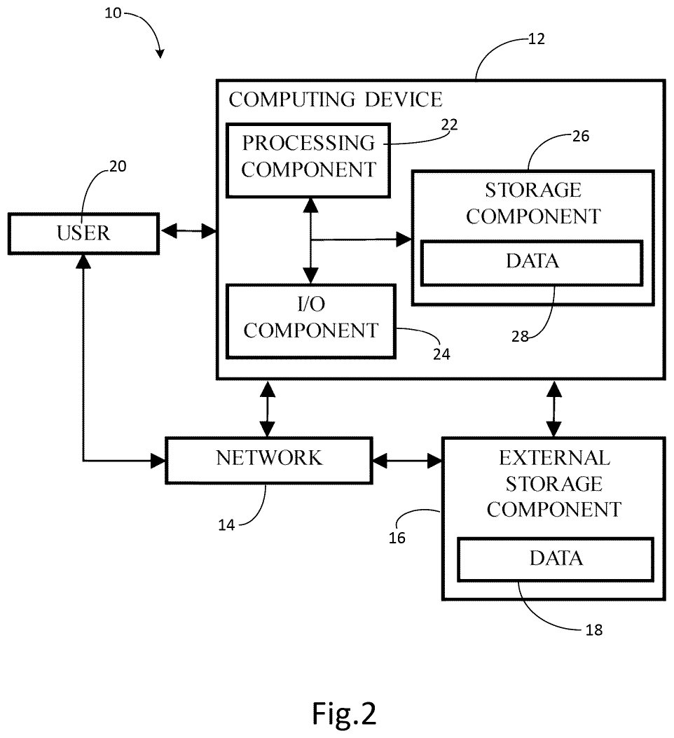

In non-limiting examples discussed herein (see FIGS. 2-8), sample spectroscopic signature(s) 108 may include and/or provide a plurality of data or information. That is, the data, for example plot points, included in each sample spectroscopic signature 108 produced or generated as a result of performing spectroscopy analysis on saliva sample 104 may aid in the detection of cognitive diseases and/or mental impairments in humans 102. In a non-limiting example where only a single portion of saliva sample 104 is analyzed, only a single set of spectroscopic signature 108 data points or information may be generated or produced (e.g., a single graph worth of plot points). In other non-limiting examples where a plurality of distinct portions of saliva sample 104 are analyzed using spectroscopy device 106, multiple, distinct sets of spectroscopic signature 108 or information may be generated or produced (e.g., a plurality of distinct graphs worth of plot points). As discussed herein, the multiple distinct sets of spectroscopic signature 108 data points may be processed separately to detect cognitive diseases/impairments in human 102 or alternatively may be averaged to form a single set of data points, prior to processing.

As shown in FIG. 1, system 100 may also include at least one computing device 110. Computing device 110 may be in operable communication with spectroscopy device 106. More specifically, computing device 110 may be connected to, in communication with, and/or operably connected with spectroscopy device 106. As a result, and during operation, computing device 110 may receive spectroscopic signature(s) 108 generated or produced by spectroscopy device 106 and may perform processes a spectroscopic signature(s) 108 to detect cognitive diseases and/or mental impairments, as discussed herein. Computing device 110 may be a stand-alone device, or alternatively, may be a portion and/or included in a larger computing device (not shown) of system 100.

For example, and as shown in FIG. 1, computing device 110 may be separate from spectroscopy device 106. Alternatively, computing device 110 may be part of the overall computing system that is used in the operation of spectroscopy device 106. As such, computing device 110 may be formed as any device and/or computing system/network that may be configured to perform the processes discussed herein to identify or detect cognitive diseases/impairments in humans. As discussed herein, computing device 110 may be configured to process spectroscopic signature(s) 108 to detect cognitive diseases or impairments using spectroscopic signature(s) 108. AS embodied in FIG. 1, computing device 110 may be in electronic communication with and/or communicatively coupled to various devices, apparatuses, and/or portions of system 100. In non-limiting examples, computing device 110 may be hard-wired and/or wirelessly connected to and/or in communication with spectroscopy device 106, and/or other components via any suitable electronic and/or mechanical communication component or technique. For example, computing device 110 may be in electronic communication with spectroscopy device 106 and neural network 112. Additionally, and as discussed herein, computing device 110 may also receive, process, and/or analyze spectroscopic signature(s) 108 during the processes discussed herein.

System 100 may also include a neural network 112. In the non-limiting example shown in FIG. 1, neural network 112 may be distinct/separate from and in communication and/or operably coupled to computing device 110. In another non-limiting example, neural network 112 may be included within and/or formed as a part of computing device 110. Neural network 112 may be any suitable component, device, program product, and/or system that may be configured to aid in the process of detecting SjD in humans 102 based on spectroscopic signature(s) 108, as discussed herein. For example, neural network 112 may be formed as any suitable machine learning device, program, and/or series of algorithms including, but not limited to, artificial/simulated neural networks including a plurality of interconnected, hidden layer nodes.

Upon receiving the plurality of spectroscopic signature(s) 108 generated/captured by spectroscopy device 106, computing device 110 using neural network 112 may perform a plurality of processes, manipulations and/or calculations using spectroscopic signature(s) 108 to detect SjD or the likelihood of SjD in humans. In the non-limiting example shown in FIG. 1, neural network 112 may utilize a predetermined statistical model 118 to process spectroscopic signature(s) 108 in order to detect SjD. That is, statistical model 118 included within neural network 112 may be used to analyze produced sample spectroscopic signature(s) 108. The predetermined statistical model 118 may be based on spectroscopic signatures 120 for a plurality modeling samples 122. In this example, spectroscopic signatures 120 for each of the plurality of modeling samples 122 are associated with one of a plurality of predetermined cognitive categories. More specifically, predetermined statistical model 118 may be built, generated, created, established, and/or based on spectroscopic signatures 120 for each of the plurality of modeling samples 122 associated with predetermined cognitive categories. In a non-limiting example, each of the predetermined spectroscopic signatures 120/modeling samples 122 provided to and/or used by neural network 112 to build predetermined statistical model 118 may have a known associated probability of the presence of SjD prior to being used by neural network 112 to form statistical model 118.

In the example, after analyzing the produced sample spectroscopic signature(s) 108 using predetermined statistical model 118, sample spectroscopic signature(s) 108 may be correlated with the presence of SjD. Sample spectroscopic signature(s) 108 may be correlated with the presence of SjD based on the spectroscopic signatures 120 for each of the plurality of modeling samples 122 of the predetermined statistical model 118, or can be used to only indicate the likelihood that SjD is present. That is, using statistical model 118, which may be generated based on spectroscopic signatures 120 for the plurality of modeling samples 122, produced sample spectroscopic signature(s) 108 may be correlated, associated, linked, and/or related to SjD. Computing device 110 using statistical model 118 may correlate sample spectroscopic signature(s) 108 by identifying human subject 102 as being associated with a predetermined likelihood for having SjD. Once identified as being associated with a likelihood, computing device 110 may subsequently detect and look for other specific evidence in human 102 in response.

In another non-limiting example, each predetermined spectroscopic signatures 120/modeling samples 122 may have an associated age or age range. As such, produced spectroscopic signature(s) 108 provided to computing device 110 may also include a known age for human 102. This in turn may increase the accuracy and/or efficiency in detecting SJD in human 102 providing saliva sample 104.

Although shown as being part of neural network 112, it is understood that predetermined statistical model 118 may also or alternatively be stored or included in computing device 110. That is, neural network 112 may generate predetermined statistical model 118 and subsequently provide statistical model 118 to computing device 110, such that statistical model 118 may be run or operate directly on computing device 110. Predetermined statistical model 118 may be generated and/or created using any suitable statistical function, operation, and/or algorithm. For example, predetermined statistical model 118 may generated and/or created using Principle component analysis (PCA), Partial Least Squares Discriminant Analysis (PLSDA), Multilayer perceptrons (MLP), Radial Basis Function (RBF), artificial neural network (ANN), support vector machine (SVM), SVM-based Discriminant analysis, or the like.

Additionally, computing device 110 may generate a report 124 related to the findings, analysis, and/or potential detection of SjD. In non-limiting examples, report 124 may be a physical print out, a graphical depiction provided on a display device of computing device 110 (e.g., screen monitor), or any other suitable visual representation providing information or data relating to the analysis of spectroscopic signature(s) 108 and/or the detection of cognitive diseases or mental impairments in human 102 providing saliva sample 104. As discussed herein, the generated report 124 may include visual information relating to the data points included, collected, and/or generated in spectroscopic signature(s) 108. Additionally, report 124 may include visual information relating the classification/correlation of the produced sample spectroscopic signature(s) 108 with the presence of SjD based on spectroscopic signatures 120 for each of the plurality of modeling samples 122 of predetermined statistical model 118.

As discussed herein, report 124 generated by the processes performed by computing device 110 and neural network 112 may aid in a physician's ability to detect SjD, or its likelihood, in humans more quickly, more accurately, in early stages of the diseases/impairment, and/or less invasively than conventional processes.

FIG. 2 depicts a schematic view of a computing environment or system (hereafter, “computing system 10”), and the various components included within computing system 10. In the non-limiting example shown in FIG. 2, computing system 10 may include at least one computing device 12 that may be configured to detect SjD in humans 102 by performing the processes discussed herein. It is understood that similarly numbered and/or named components may function in a substantially similar fashion.

The computing system shown in FIG. 2 may include any type of computing device(s) and for example includes at least one processor or processing component(s) 22, storage component 26 with data 28, input/output (I/O) component(s) 24 (including a keyboard, touchscreen, or monitor display), and a communications pathway. In general, processing component(s) 22 execute program code which is at least partially fixed or stored in storage component 26. While executing program code, processing component(s) 22 can process data 28, which can result in reading and/or writing transformed data from/to storage component 26 and/or I/O component(s) 24 for further processing. The pathway provides a communications link between each of the components and computing device(s). I/O component 24 can comprise one or more human I/O devices, which enables user to interact with computing device(s) to analyze produced sample spectroscopic signatures and detect SjD in humans, as discussed herein. Computing device(s) 12 may also be implemented in a distributed manner such that different components reside in different physical locations.

Storage components may also include modules, data and/or electronic information relating to various other aspects of computing system 10. Specifically, operational modules, electronic information, and/or data relating to saliva sample data, spectroscopic analysis (e.g., Raman spectroscopy) data, spectroscopic signature data, predetermined statistical model data, predetermined cognitive categories data, biomolecules data, and/or correlating data. The operational modules, information, and/or data may include the required information and/or may allow computing system 10, and specifically computing device 12, to perform the processes discussed herein for detecting SjD in humans.

Computing system 10, and specifically computing device 12 of computing system, may also be in communication with external storage component 16. External storage component 16 may be configured to store various modules, data 18 and/or electronic information relating to various other aspects of computing system 10, similar to storage component of computing device(s) 12. Additionally, external storage component 16 may be configured to share (e.g., send and receive) data and/or electronic information with computing device(s) of computing system. In the non-limiting example shown in FIG. 2, external storage component 16 may include any or all of the operational modules and/or data 18 shown to be stored on storage component 26. Additionally, external storage component 16 may also include a secondary database that user 20 may interact with, provide information/data to, and/or may include information/data relating to diagnosis. In a non-limiting example, external storage component may be a cloud-based storage component or system. In other non-limiting examples, external storage component may also include and/or be in communication with a neural network to aid in computation and/or data processing as discussed herein.

In a non-limiting example shown in FIG. 2, computing device(s) may be in communication with and/or may be configured to share (e.g., send and receive) data and/or electronic information over a network 14. Network 14 may represent a closed network, such as a local area network (LAN) or may include the internet. Network 14 may also include secondary database including similar data as storage component 26, and/or may include or be in communication with a neural network 112 to aid in computation and/or data processing as discussed herein.

A demonstrative study was performed to show that Raman hyperspectroscopy of saliva and machine learning for differentiating SjD patients from healthy control (HC) individuals and individuals treated with radiation therapy for head and neck cancers (RD), as these patients also suffer from salivary hypofunction and xerostomia. It was demonstrated the effectiveness of using Raman hyperspectroscopy to differentiate between SjD, HC, and RD patients using a rapid, noninvasive saliva test.

Saliva samples (one sample per donor) were collected from 72 individuals representing HC, SjD, and RD at Albany Medical Center (AMC, Albany, NY) in accordance with the approved protocol of the AMC institutional review board (IRB). Nine randomly selected samples were set aside for external validation. The 63 remaining samples were used as a training dataset for a classification model. Thirty-six spectra were collected from each saliva sample using an automatic mapping technique.

Raman hyperspectroscopy takes advantage of a microheterogeneity of dry saliva to acquire information about various biochemical components, including those with a relatively low concentration, such as disease biomarkers. Machine learning analysis of the Raman hyperspectral datacube (two spatial coordinates and a Raman spectrum) allows for developing a spectral signature of the disease, potentially including contributions from multiple biomarkers that can be used for disease diagnostics.

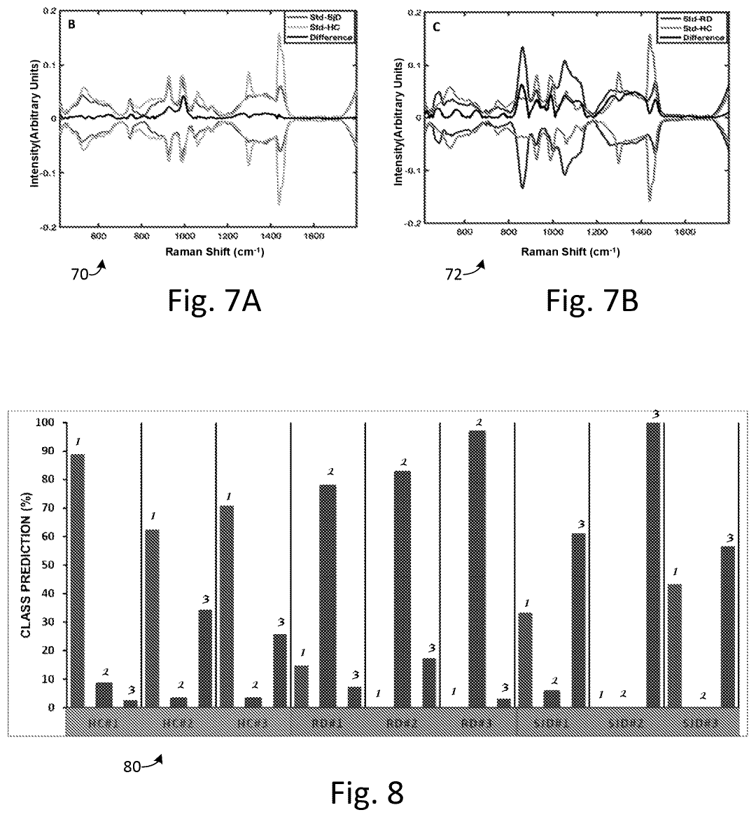

Mean preprocessed Raman spectra calculated for each class of donors including HC, RD, and SjD, are shown in FIG. 6. FIG. 6 is a graph of the pre-processed mean Raman spectra of saliva acquired from Healthy controls (HC), Radiation therapy patients (RD) and Sjogren syndrome patients (SjD). Areas selected by a Genetic Algorithm are highlighted (transparent grey). The spectra depict the biochemical composition of saliva with characteristic peaks and peak assignments listed in Table-2A. There are noticeable variations between the mean spectra in FIG. 6. Yet, the difference spectrum between SjD and HC mean spectra is within one standard spectral deviation of SjD and HC classes (FIG. 7A).

FIG. 7A is a graph 70 illustrating a difference spectrum between SjD and HC mean spectra, and one standard spectral deviation of SjD and HC spectra. FIG. 7B is a graph 72 illustrating a difference spectrum between mean spectra of RD and HC, and standard spectral deviation of RD and HC. Similarly, the difference spectrum between RD and HC spectra also remains within one standard deviation (FIG. 7B). The latter means that the variations between the mean spectra are statistically insignificant.

Consequently, the changes in the intensity of individual Raman bands cannot be used directly for spectrum classification. Instead, statistical analysis based on their entire spectra or significant parts is required. This is not a surprising result as saliva biochemical composition might vary significantly because of environment, diet, and medical conditions making spectral changes specific to the disease subtle.

Supervised multivariate analysis, including machine learning algorithms, can identify these multiple small but specific differences between spectral signatures and build diagnostic classification models based on them.

A type of supervised multivariate analysis, the Partial least squares-discriminant analysis (PLS_DA) model, was built to determine the number of latent variables and data distribution. Selecting an optimal number of latent variables improves the model's interpretability and reduces the risk of overfitting. The PLS toolbox offers an outliers removal technique for the PLS_DA model called T2 Hotelling. We used hoteling T2 scores with conservative statistical threshold determined by PLS_DA to eliminate four outliers' samples, including 2-HC and 2-RD (FIG. S1, supplementary information). The final calibration dataset consisted of 59 donors with 1878 spectra and was introduced to GA (Genetic algorithm) for feature selection.

The Raman spectral dataset with many spectra per class is a high-dimensional dataset with various features. Feature selection techniques like Genetic Algorithm (GA) can reduce dimensionality by selecting only spectral components that show significant variations between classes of the dataset. We employed the advanced machine learning classification technique Support vector machine-discriminant analysis (SVM_DA) to analyze collected spectral data for inter-class differences. SVM_DA selects the area (data points) of the spectra specific to each class with the help of GA and generates a hyperplane (separating line) between classes for classification. Tentative assignments of important Raman bands selected by GA are available in FIG. 4.

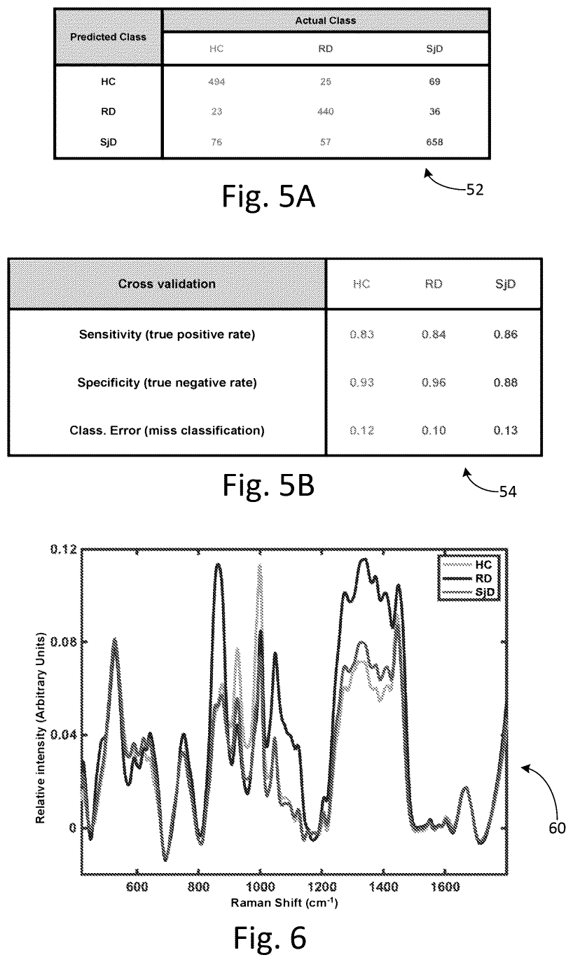

FIG. 4 is a table 42 with the assignments of the main Raman bands of saliva based on literature data. Raman bands selected by Genetic Algorithm are highlighted with grey color. We imported the calibration spectral dataset created by GA into the SVM_DA model for training consisting of 1878 total spectra labeled with their respective classes. We used 11 LVs selected using PLS_DA to train an SVM_DA classification model. Next, we applied the Venetian blind cross-validation with 50 splits and 20 spectra in each division. The Venetian Blind method can help to ensure that the build SVM_DA model is not overfitted. The SVM-DA model offered cross-validation sensitivity (true positive rate) of 86% for SjD (FIG. 5A) at a spectral level. FIG. 5A is a table 52 of the cross-validation predictions for individual spectra collected for samples in the calibration dataset. We collected 36 spectra per sample to represent sample heterogeneity. A 97% accuracy at the sample level (2 samples from 63 were misclassified) was achieved by SVM_DA using a 50% threshold since the majority of spectra were correctly assigned to their actual class.

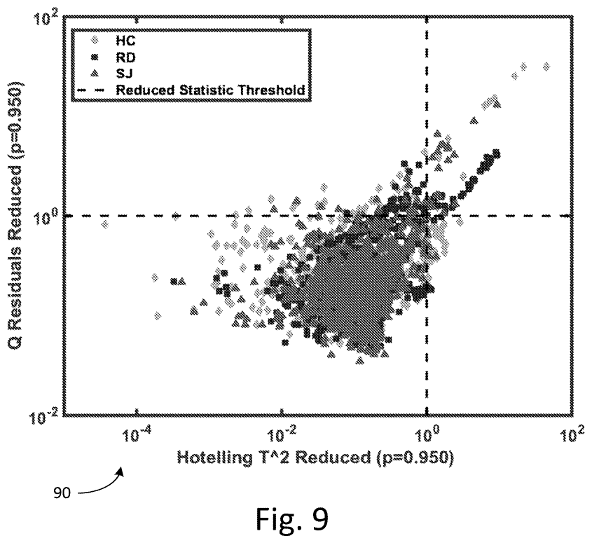

FIG. 5B is a table 54 of the performance matrix of SVM_DA cross validation at spectral level. The ultimate test for the validity of a classification model is its external validation based on samples not included in the training dataset. We performed the external validation of the SVM_DA model using nine samples that were not used to create the model. The confusion matrix revealed that an SVM_DA model showed 79% accuracy at the spectral level, with some spectra assigned to incorrect classes (Table-S1A, supplementary information SI). The prediction of nine external validation samples is summarized in FIG. 8 and FIG. 9. FIG. 8 is a graph 80 of the external validation of the SVM_DA model. The percent spectra assigned to HC (1), RD (2), and SjD (3) classes are reported for individual samples. The histogram shows that all nine samples were assigned to their actual class by the SVM_DA model at the 50% threshold.

FIG. 9 is a graph 90 illustrating the outlier removal using Hoteling T2. The spectra with the T2 Hotteling values exceeding reduced statistical threshold (--) considered outliers (Grey area top right). Note that the model can not only successfully differentiate between Sjogren disease patient saliva and healthy saliva, but it can also distinguish between radiation therapy patient saliva and Sjogren disease patient saliva.

The present study demonstrated that Raman hyperspectroscopy combined with machine learning can successfully differentiate patients with Sjogren's disease from head and neck cancer radiation patients and healthy individuals on the basis of a non-invasive saliva test. While the investigation was carried out utilizing a constrained sample size of 72 patients, it is noteworthy that accurate predictions were achieved across all nine external validation samples. Raman spectra were collected from multiple (36) spots on heterogeneous dry saliva samples to increase the probability of detecting specific disease biomarkers, which are typically present at a low concentration. A single reading from the sample is insufficient, and multiple readings and full spectral-level predictions are required to make the final classification at the donor level. In our earlier study, the development of Alzheimer's disease from the mild to moderate stage increased the amount of specific disease biomarkers in blood and, as a result, significantly increased the portion of individual Raman spectra in the hyperspectral datacube, which were characteristic of the disease.

Raman hyperspectroscopy is ideal for disease diagnostic tests due to its non-invasive nature, rapid analysis, and high sensitivity in detecting molecular changes associated with various diseases. The abundance of biomolecules in saliva allows for the identification of potential disease biomarkers, while the cost-effectiveness and portability of Raman spectroscopy make it feasible for point-of-care applications and resource-limited settings, enabling early disease detection and monitoring.

Raman hyperspectroscopy of saliva holds great promise for development of a noninvasive, efficient, rapid, and inexpensive diagnostic test for Sjogren syndrome. Due to the noninvasive nature of the test, it could be used to screen patients for participation in clinical trials and follow disease progression or response to treatment. It might also be useful to identify early stages of disease development; however, further work will be required to determine at what stage the disease can be detected with Raman hyperspectroscopy. While the inventors have shown the ability to distinguish between SjD and radiation-induced xerostomia, it should be apparent to one of skill in the art that testing more samples can validate further the developed model's sensitivity as well as its selectivity relative to other diseases. This method could have broader applicability for those patients with radiation to the head and neck. For these patients, spectral analysis should be correlated with specific radiation doses and used to track saliva quality over time . . .

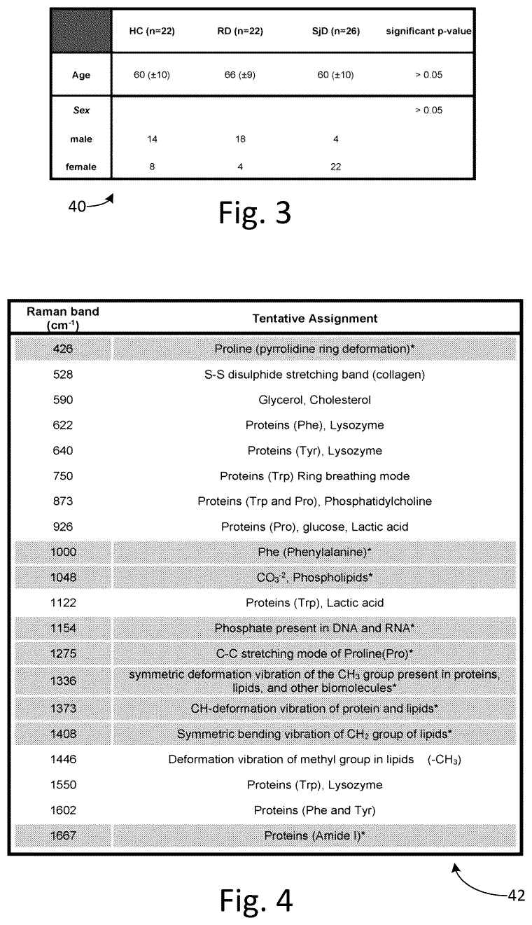

In the present study, saliva samples were collected from 72 donors (one sample per donor, 24-HC, 26-SjD, 22-RD), at Albany College of Medicine under approval of the institutional review board (IRB) and stored at −20° C. Age information about the donors can be found in FIG. 3. FIG. 3 is a table 40 of the information about the donors' age and sex for Healthy control (HC), Radiation (RD), and Sjogren syndrome patients (SjD). Samples were thawed and centrifuged for 5 mins at 20,000 rpm. The supernatant was collected and used for the Raman spectral analysis. About 10 μL of saliva supernatant was deposited on an aluminum foil-covered glass slide and allowed to dry overnight. The aluminum foil minimizes substrate interference.

All Raman spectra were collected using a Horiba Xplora-Plus Raman microscope (HORIBA Scientific). The PRIOR automatic mapping stage was used to collect Raman spectra from multiple locations on dry saliva samples using a SOX objective to probe the sample heterogeneity and generate the hyperspectral datacube. Spectra were recorded in the range of 400-1800 cm-1 using a 785-nm laser source with 100% power (110 mW). A total of 36 spectra per sample were collected with a 30-s acquisition time at each location and three accumulations at each location using LabSpec6 software.

A total of 2264 spectra from 72 saliva samples were imported into MATLAB (R2019b) programming software (MathWorks, Inc) equipped with PLS-Toolbox 9.0 (2021) (Eigenvector Research, Inc., Manson, WA USA 98831). Raman spectra with extensive cosmic rays or low signal-to-noise ratio were removed from the dataset. Nine samples (3-HC,3-SjD,3-RD) were randomly selected and set aside for external validation. We applied the same automatic preprocessing procedure to all spectra, including baseline correction (weighted least square-6), normalization by 1667-cm-1 band, and smoothing (Sav Gol filter width 31, order-5). This band, which was tentatively assigned to protein Amid I vibrational mode, showed the least changes among strong Raman bands in saliva spectra. We assigned classes to each spectrum in the training dataset as HC, SjD, and RD. Next, we applied feature selection techniques genetic algorithm (GA) to select spectral components from the training dataset. The parameters of GA are given as follows: the population size was set to 62, the mutation rate to 0.005, and the maximum number of generations for every run was set to 100. We used double cross-over breeding with a window width of 30%. We ran GA 100 times independently to select diagnostic feature information from the measured Raman spectra of the calibration dataset. We employed a SVM_DA classification model using the PLS toolbox extension of MATLAB. The features of the training dataset were optimized using a Genetic Algorithm (GA) prior to constructing the model.

With reference again to FIGS. 1 and 2, the present invention can provide, in one embodiment, a system 100 for detecting Sjogren's Syndrome disease (SjD) including a spectroscopy device 106 subjecting at least a portion of a saliva sample 104 from a human 102 to a spectroscopic analysis to produce a sample spectroscopic signature (e.g. FIG. 6) for the saliva sample 104. There is at least one computing device 12 in operable communication with the spectroscopy device 106, the at least one computing device 12 configured to detect SjD in the human subject 102 by analyzing the produced sample, here a saliva sample 104, and its spectroscopic signature using a predetermined statistical model, such as that shown in FIG. 9. The predetermined statistical model is based on spectroscopic signatures for a plurality modeling samples, as is described herein. The spectroscopic signatures for each of the plurality of modeling samples are associated with SjD, the computing device 12 correlates the produced sample spectroscopic signature (FIG. 6) with SjD based on the spectroscopic signatures for each of the plurality of modeling samples of the predetermined statistical model.

The computing device 12 can be further configured to determine a likelihood of SjD in the human subject, e.g. the likelihood of the presence of SjD as opposed to a binary yes/no. The computing device 12 can also correlate the produced sample (saliva sample 104) with a spectroscopic signature further by identifying the human subject 102 as being associated with the likelihood of SjD, and detecting SjD in the human subject 102 with the association with the likelihood of SjD. Accordingly, the weighted likelihood could also be used to give a binary decision of yes/no.

Further, the spectroscopy device 106 (FIG. 1) can subject at least the portion of the saliva sample 104 to the spectroscopic analysis by performing spectroscopy on at least the portion of the saliva sample 104, the spectroscopy selected from the group consisting of: near-infrared (NIR) Raman spectroscopy, Raman microspectroscopy, Surface Enhanced Raman spectroscopy (SERS), surface enhanced resonance Raman spectroscopy (SERRS), Raman hyper spectroscopy, Fourier transform Raman spectroscopy, IR absorption spectroscopy, Fourier Transform Infrared absorption (FTIR), Attenuated Total Reflection (ATR) FTIR, IR reflection spectroscopy, vibrational spectroscopy, and coherent anti-Stokes Raman Spectroscopy (CARS). The spectroscopy device 106 can also subject at least the portion of the saliva sample 104 to the spectroscopic analysis by exposing biomolecules of the saliva sample 104 to a spectroscopic analysis, the biomolecules including at least one of structural properties, conformational properties, or compositional variations that define the produced sample spectroscopic signature for the saliva sample, and the biomolecules can include at least one of proteins, lipids, peptides, amino acids, electrolytes, mucus, enzymes, or antibacterial species. Additionally, the spectroscopy device 106 can subject at least a portion of the saliva sample 104 to the spectroscopic analysis by subjecting a plurality of portions of the saliva sample 104 to the spectroscopic analysis to produce a plurality of distinct sample spectroscopic signatures for the saliva sample, with each of the plurality of portions positionally distinct from the others in the saliva sample 104.

The system can be embodied with the computing device 12 analyzing the produced sample spectroscopic signature using the predetermined statistical model by analyzing each of the plurality of the produced sample spectroscopic signatures using the predetermined statistical model (such as shown in FIG. 9), and correlating the produced sample spectroscopic signature with a presence of SjD by correlating each of the plurality of produced sample spectroscopic signatures based on the spectroscopic signatures for each of the plurality of modeling samples of the predetermined statistical model. Further, the computing device 12 can be configured to detect SjD in the human subject 102 further by determining a final, predetermined SjD diagnosis based on each of the plurality of correlated, produced sample spectroscopic signatures.

In an embodiment, with general reference to FIGS. 1 and 2, in the invention can provide a method for detecting SjD) that starts with providing a biological sample, such as saliva sample 104, from a human subject 102, then subjecting at least a portion of the saliva sample 104 to a Raman hyperspectroscopic analysis to produce a sample spectroscopic signature for the saliva sample 101, then analyzing the produced sample spectroscopic signature using a predetermined statistical model (such as in FIG. 9), the analysis occurring on the computer device 12. The predetermined statistical model is based on spectroscopic signatures for a plurality of modeling samples and the spectroscopic signatures for each of the plurality of modeling samples are associated with SjD. The method then includes correlating the produced sample spectroscopic signature with a presence of SjD based on the spectroscopic signatures for each of the plurality of modeling samples of the predetermined statistical model (FIG. 9).

The method can include determining a likelihood of the presence of SjD, e.g. such as the probability of the presence of SjD as opposed to a binary yes/no on diagnosis. The correlating of the produced sample spectroscopic signature can further includes identifying the human subject 102 as being associated with a predetermined likelihood of the presence SjD, and then detecting the presence of SjD based upon the predetermined likelihood of SjD. The subjecting of at least the portion of the biological sample, such as saliva sample 104, to the spectroscopic analysis further includes performing Raman hyperspectroscopy on at least the portion of the saliva sample 104, with the Raman hyperspectroscopy including one of the group of near-infrared (NIR) Raman spectroscopy, Raman microspectroscopy, Surface Enhanced Raman spectroscopy (SERS), surface enhanced resonance Raman spectroscopy (SERRS), Fourier transform Raman spectroscopy, and coherent anti-Stokes Raman Spectroscopy (CARS).

The subjecting of at least the portion of the saliva sample 104 to the spectroscopic analysis can further include exposing biomolecules of the saliva sample 104 to a spectroscopic analysis, the biomolecules including at least one of structural properties, conformational properties, or compositional variations that define the produced sample spectroscopic signature for the saliva sample 104. Note that the biomolecules include at least one of: proteins, lipids, peptides, amino acids, electrolytes, mucus, enzymes, or antibacterial species. Subjecting at least the portion of the saliva sample 104 to the spectroscopic analysis can further include subjecting a plurality of portions of the saliva sample 104 to the spectroscopic analysis to produce a plurality of distinct sample spectroscopic signatures for the saliva sample 104, with each of the plurality of portions positionally distinct from the others in the saliva sample.

In an embodiment, the analyzing of the produced sample spectroscopic signature using the predetermined statistical model further includes analyzing each of the plurality of the produced sample spectroscopic signatures using the predetermined statistical model (FIG. 9) and correlating of the produced sample spectroscopic signature with SjD can further include correlating each of the plurality of produced sample spectroscopic signatures with the presence of SjD based on the spectroscopic signatures for each of the plurality of modeling samples of the predetermined statistical model. Determining a diagnosis of SjD can be based upon on each of the plurality of correlated, produced sample spectroscopic signatures.

The method can further include discarding predetermined portions of the produced sample spectroscopic signature prior to the analyzing of the produced sample spectroscopic signature using the predetermined statistical model, where the discarded predetermined portions of the produced sample spectroscopic signature are inconclusive for correlating the produced sample spectroscopic signature with spectroscopic signatures for the presence of SjD. Here, the produced sample spectroscopic signature for the saliva sample 104 can include a vibrational signature of the provided saliva sample 104.

Aspects of the present invention are described herein with reference to flowchart illustrations and/or block diagrams of methods, apparatus (systems), and/or computer program products according to embodiments of the invention. It will be understood that each block of the flowchart illustrations and/or block diagrams, and combinations of blocks in the flowchart illustrations and/or block diagrams, can be implemented by computer readable program instructions.

It is understood that computing device(s) may be implemented as a computer program product stored on a computer readable storage medium. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A method for detecting Sjogren's Syndrome disease (SjD), the method comprising:
providing a biological sample from a human subject;
subjecting at least a portion of the biological sample to a Raman hyperspectroscopic analysis to produce a sample spectroscopic signature for the biological sample;
analyzing the produced sample spectroscopic signature using a predetermined statistical model, the predetermined statistical model based on spectroscopic signatures for a plurality of modeling samples, wherein the spectroscopic signatures for each of the plurality of modeling samples are associated with SjD; and
correlating the produced sample spectroscopic signature with a presence of SjD based on the spectroscopic signatures for each of the plurality of modeling samples of the predetermined statistical model.

2. The method of claim 1, further including determining a likelihood of the presence of SjD.

3. The method of claim 2, wherein correlating of the produced sample spectroscopic signature further includes:
identifying the human subject as being associated with a predetermined likelihood of the presence SjD; and
detecting the presence of SjD based upon the predetermined likelihood of SjD.

4. The method of claim 1, wherein:
the biological sample is a saliva sample; and
subjecting of at least the portion of the saliva sample to the spectroscopic analysis further includes:
performing Raman hyperspectroscopy on at least the portion of the saliva sample, the Raman hyperspectroscopy including one of the group consisting of:
near-infrared (NIR) Raman spectroscopy, Raman microspectroscopy, Surface Enhanced Raman spectroscopy (SERS), surface enhanced resonance Raman spectroscopy (SERRS), Fourier transform Raman spectroscopy, and coherent anti-Stokes Raman Spectroscopy (CARS).

5. The method of claim 1, wherein the subjecting of at least the portion of the biological sample to the spectroscopic analysis further includes:
exposing biomolecules of the biological sample to a spectroscopic analysis, the biomolecules including at least one of structural properties, conformational properties, or compositional variations that define the produced sample spectroscopic signature for the biological sample.

6. The method of claim 5, wherein the biomolecules include at least one of:
proteins, lipids, peptides, amino acids, electrolytes, mucus, enzymes, or antibacterial species.

7. The method of claim 1, wherein the subjecting at least the portion of the biological sample to the spectroscopic analysis further includes:
subjecting a plurality of portions of the biological sample to the spectroscopic analysis to produce a plurality of distinct sample spectroscopic signatures for the biological sample, each of the plurality of portions positionally distinct from others in the biological sample.

8. The method of claim 7, wherein:
the analyzing of the produced sample spectroscopic signature using the predetermined statistical model further includes:
analyzing each of the plurality of the produced sample spectroscopic signatures using the predetermined statistical model; and
correlating of the produced sample spectroscopic signature with a presence of SjD based on the spectroscopic signatures for each of the plurality of modeling samples of the predetermined statistical model.

9. The method of claim 8, further comprising:
determining a diagnosis of SjD based upon on each of the plurality of correlated, produced sample spectroscopic signatures.

10. The method of claim 1, further comprising discarding predetermined portions of the produced sample spectroscopic signature prior to the analyzing of the produced sample spectroscopic signature using the predetermined statistical model, wherein the discarded predetermined portions of the produced sample spectroscopic signature are inconclusive for correlating the produced sample spectroscopic signature with spectroscopic signatures for the presence of SjD.

11. The method of claim 1, wherein the produced sample spectroscopic signature for the biological sample includes a vibrational signature of the provided biological sample.

12. A system for detecting Sjogren's Syndrome disease (SjD), comprising:
a spectroscopy device subjecting at least a portion of a biological sample from a human to a spectroscopic analysis to produce a sample spectroscopic signature for the biological sample; and
at least one computing device in operable communication with the spectroscopy device, the at least one computing device configured to detect SjD in the human subject by:
analyzing the produced sample spectroscopic signature using a predetermined statistical model, the predetermined statistical model based on spectroscopic signatures for a plurality modeling samples, wherein the spectroscopic signatures for each of the plurality of modeling samples are associated with SjD; and
correlating the produced sample spectroscopic signature with SjD based on the spectroscopic signatures for each of the plurality of modeling samples of the predetermined statistical model.

13. The system of claim 12, wherein the at least one computing device further configured to determine a likelihood of SjD in the human subject.

14. The system of claim 13, wherein the at least one computing device correlates the produced sample spectroscopic signature further by:
identifying the human subject as being associated with the likelihood of SjD; and
detecting SjD in the human subject with the association with the likelihood of SjD and.

15. The system of claim 12, wherein the spectroscopy device subjects at least the portion of the biological sample to the spectroscopic analysis by performing spectroscopy on at least the portion of the biological sample, the spectroscopy selected from the group consisting of:
near-infrared (NIR) Raman spectroscopy, Raman microspectroscopy, Surface Enhanced Raman spectroscopy (SERS), surface enhanced resonance Raman spectroscopy (SERRS), Raman hyper spectroscopy, Fourier transform Raman spectroscopy, IR absorption spectroscopy, Fourier Transform Infrared absorption (FTIR), Attenuated Total Reflection (ATR) FTIR, IR reflection spectroscopy, vibrational spectroscopy, and coherent anti-Stokes Raman Spectroscopy (CARS).

16. The system of claim 12, wherein the spectroscopy device subjects at least the portion of the biological sample to the spectroscopic analysis by exposing biomolecules of the biological sample to a spectroscopic analysis, the biomolecules including at least one of:
structural properties, conformational properties, or compositional variations that define the produced sample spectroscopic signature for the biological sample; and
wherein the biomolecules include at least one of: proteins, lipids, peptides, amino acids, electrolytes, mucus, enzymes, or antibacterial species.

17. The system of claim 12, wherein:
the biological sample is saliva; and
the spectroscopy device subjects at least the portion of the saliva sample to the spectroscopic analysis by subjecting a plurality of portions of the saliva sample to the spectroscopic analysis to produce a plurality of distinct sample spectroscopic signatures for the saliva sample, each of the plurality of portions positionally distinct from others in the saliva sample.

18. The system of claim 17, wherein the at least one computing device analyzes the produced sample spectroscopic signature using the predetermined statistical model by:
analyzing each of the plurality of the produced sample spectroscopic signatures using the predetermined statistical model; and
correlates the produced sample spectroscopic signature with a presence of SjD by correlating each of the plurality of produced sample spectroscopic signatures based on the spectroscopic signatures for each of the plurality of modeling samples of the predetermined statistical model.

19. The system of claim 18, wherein the at least one computing device configured to detect SjD in the human subject further by determining a final, predetermined diagnosis of SjD based upon produced sample spectroscopic signatures.

20. A system for detecting Sjogren's Syndrome disease (SjD), comprising:
a spectroscopic means for subjecting at least a portion of a biological sample from a human to a spectroscopic analysis to produce a sample spectroscopic signature for the biological sample; and
a computing means in operable communication with the spectroscopy means, the computing means for detecting SjD in the human subject by:
analyzing the produced sample spectroscopic signature using a predetermined statistical model, the predetermined statistical model based on spectroscopic signatures for a plurality modeling samples, wherein the spectroscopic signatures for each of the plurality of modeling samples are associated with SjD; and
correlating the produced sample spectroscopic signature with SjD based on the spectroscopic signatures for each of the plurality of modeling samples of the predetermined statistical model.
